(19) United States
(12) Patent Application Publication
(10) Pub. No.: US 2025/0229965 A1
(43) Pub. Date: Jul. 17, 2025

(54) BLISTER PACK CONFIGURED TO HOLD AND/OR PREVENT INADVERTENT ACTUATION OF A DEVICE FOR ADMINISTERING A DRUG

(71) Applicant: CSP Technologies, Inc., Auburn, AL (US)

(72) Inventors: Franklin Lee LUCAS, JR., Auburn, AL (US); Ethan Ross PERDUE, Auburn, AL (US); Danielle MINTON, Auburn, AL (US)

(21) Appl. No.: 19/026,216

(22) Filed: Jan. 16, 2025

Related U.S. Application Data
(60) Provisional application No. 63/621,646, filed on Jan. 17, 2024.

(51) Int. Cl. B65D 75/36; A61K 9/00; A61K 31/137; A61K 31/485; A61K 38/26; A61M 15/00; A61M 15/08; B65D 75/56

(52) U.S. Cl. CPC B65D 75/366; A61K 9/0073; A61K 31/137; A61K 31/485; A61K 38/26; A61M 15/0043; A61M 15/08; B65D 75/563; A61M 2209/06; A61M 2210/0618

(57) ABSTRACT

A blister pack designed to hold a device for administering a drug includes a cover. The cover defines a cavity to at least partially surround the device for administering the drug. The cavity is formed by injection molding the cover. A backing is placed on the rear surface of the cover and forms a seal with the cover, storing the device for administering the drug in a closed environment.

### CROSS-REFERENCE TO RELATED APPLICATION

This application claims priority to U.S. Provisional Patent Application No. 63/621,646, filed Jan. 17, 2024, and titled “BLISTER PACK CONFIGURED TO HOLD AND/OR PREVENT INADVERTENT ACTUATION OF A DEVICE FOR ADMINISTERING A DRUG,” the disclosure of which is hereby incorporated by reference in its entirety.

### FIELD

The presently disclosed technology relates generally to packaging configured to hold and preferably prevent inadvertent opening and/or actuation of a device designed to administer contents, such as but not limited to a drug. More particularly, in an optional aspect, the presently disclosed technology relates to a blister pack configured to hold and preferably prevent inadvertent opening thereof and/or actuation of an inhaler, e.g., nasal inhaler device, therein.

### BACKGROUND

Devices for administering or releasing contents, such as drugs, are often stored or transported in blister packs. For example, medications administered in the form of oral solid dosage forms, are often stored in blister packs.

Prior art blister packs can, from time to time and/or in a particular circumstance or use, be crushed or punctured, potentially causing the blister pack to inadvertently open. This is due to the generally flexible construction of such packaging. While a blister configuration could be desirable to potentially house a drug delivery device, inadvertent opening of the blister pack can unnecessarily or undesirably expose the device(s) therein to the external environment. Such exposure risks wasting or degrading the contents of the devices (e.g., in the event that the drug in a delivery device degrades in the presence of moisture) or damaging or misplacing the device(s). This is particularly problematic when the drug is either expensive or difficult to procure, or when the user may have difficulty quickly or reliably accessing replacement medicine needed to treat a dire medical situation.

### SUMMARY

In an effort to overcome the above-described and other drawbacks of the prior art, Applicant has developed improved systems, mechanisms and methods for storing drug delivery devices to prevent damage and inadvertent opening of the blister pack.

In one object, the presently disclosed technology is directed to a blister pack comprising a cover. The cover comprises a cavity formed therein, the cavity configured to at least partially receive a drug delivery device, such as a single dose nasal inhaler containing a nasally administered medication. The cavity is formed in the cover by injection molding. A backing is disposed on the rear surface of the body, forming a seal with the body to protect the contents of the blister pack.

In another aspect, the body is made of a particularly rigid construction, such as to prevent crushing, puncturing, or other damage to the drug delivery device stored in the blister pack. The body may be made of a polypropylene or polyethylene material, and/or may comprise a thickness between 1 mm and 2 mm to provide enhanced structural protection.

### BRIEF DESCRIPTION OF THE DRAWINGS

The following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a front diagrammatic view of an optional nasal inhaler device that may be stored in a blister pack according to the disclosed concept, shown in its rest position before actuation.

FIG. 2 is a front diagrammatic view of the nasal inhaler device of FIG. 1, showing the device at the start of actuation.

FIG. 3 is a front diagrammatic view of the nasal inhaler device of FIG. 1, showing the device in its fully actuated or depressed position.

FIG. 4 is a front perspective view of a blister pack according to an optional aspect of the presently disclosed technology;

FIG. 5 is a front view of the blister pack of FIG. 1.

FIG. 6 is a rear perspective view of the blister pack of FIG. 1.

FIG. 7 is a side view of the blister pack of FIG. 1.

FIG. 8 is a cross-sectional view of the blister pack of FIG. 1, along section line 8-8 in FIG. 5, showing the device of FIG. 1 stored inside, the blister pack and device together forming a medication delivery system.

FIG. 9 is a front perspective view of a blister pack according to an optional aspect of the presently disclosed technology;

FIG. 10 is a front view of the blister pack of FIG. 9.

FIG. 11 is a side view of the blister pack of FIG. 9.

FIG. 12 is a cross-sectional view of the blister pack as shown in FIG. 9, along section line 12-12 in FIG. 11, showing the device of FIG. 1 stored inside, the blister pack and device together forming a medication delivery system.

### DETAILED DESCRIPTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the word “may” is used in a permissive sense (i.e., meaning having the potential to) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

As used herein, “and/or” means that either or both of the items separated by such terminology are involved. For example, the phrase “A and/or B” would mean A alone, B alone, or both A and B.

As used herein, “generally” means “in a general manner” relevant to the term being modified as would be understood by one of ordinary skill in the art.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

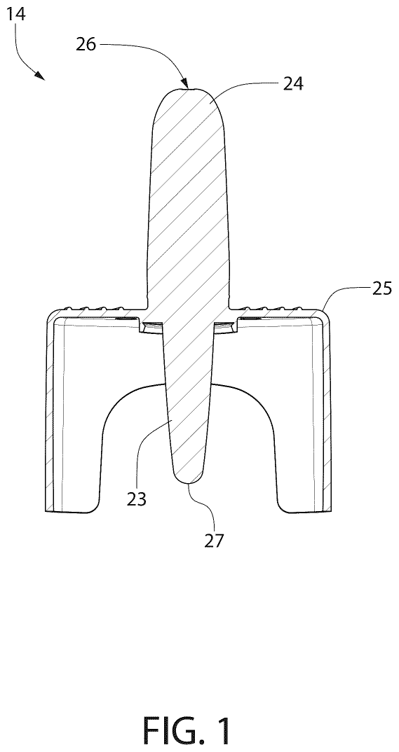

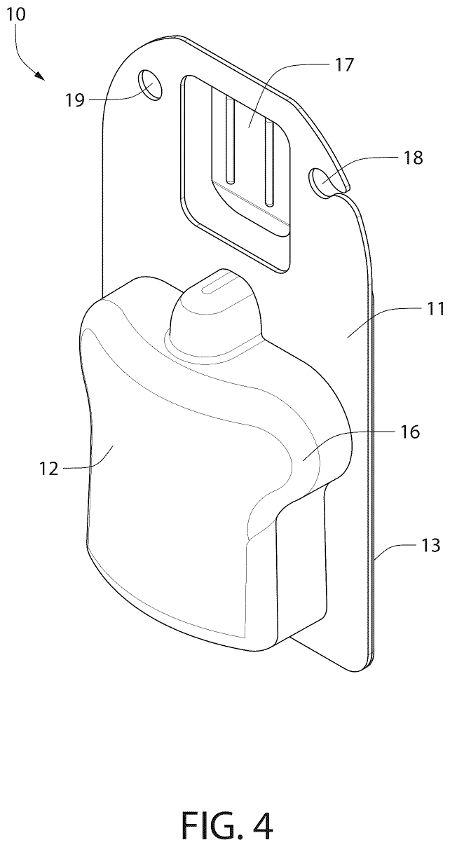

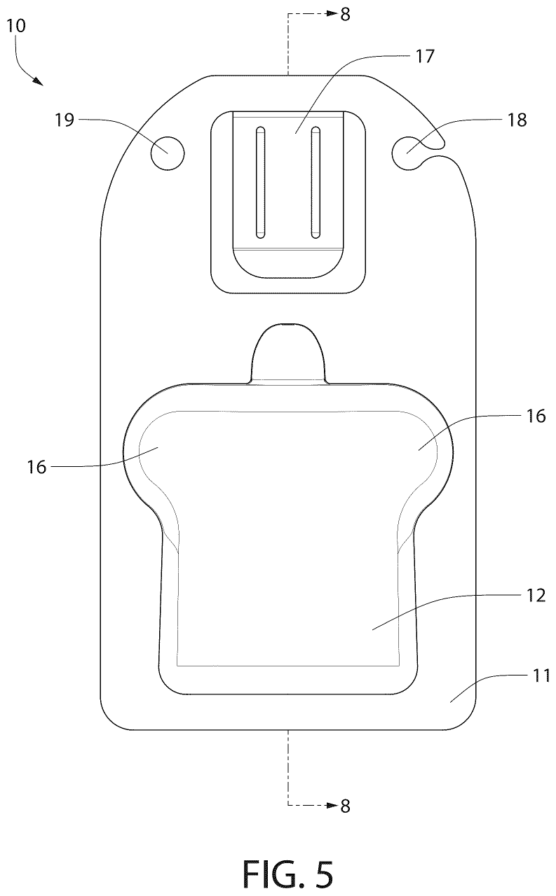

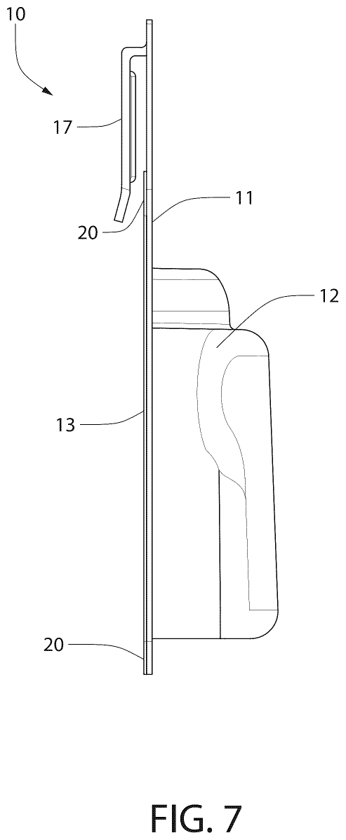

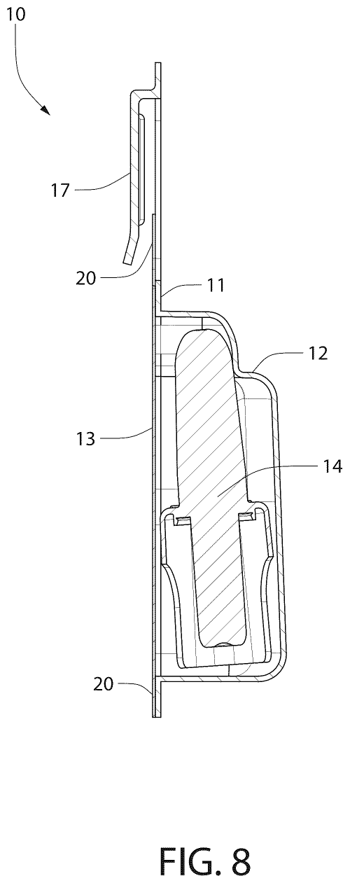

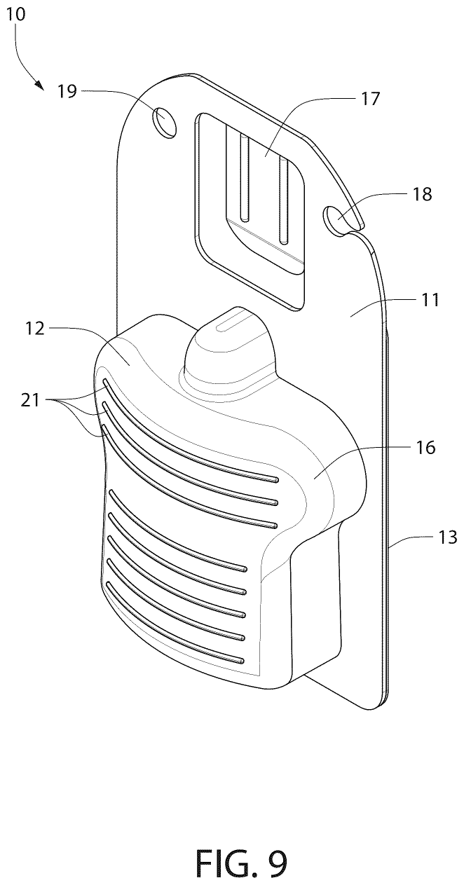

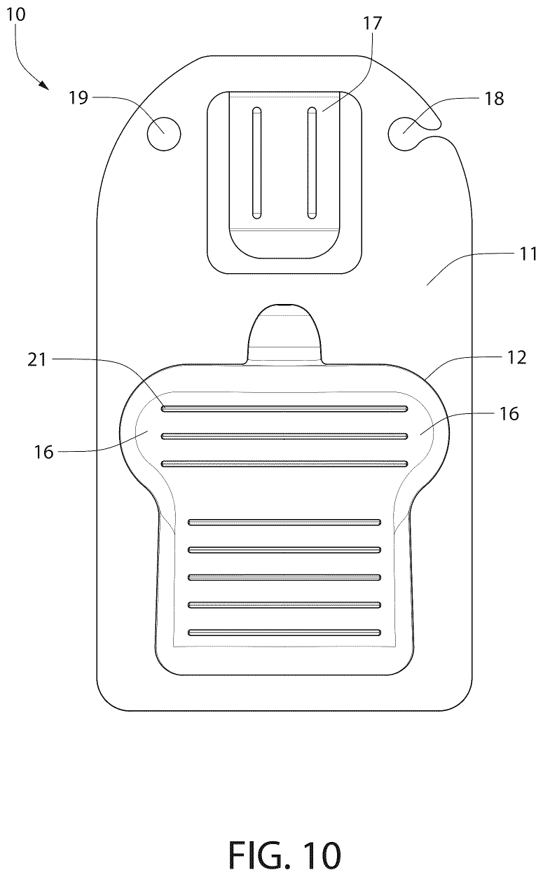

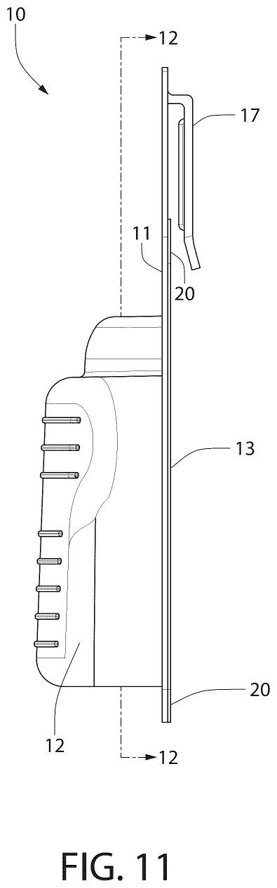

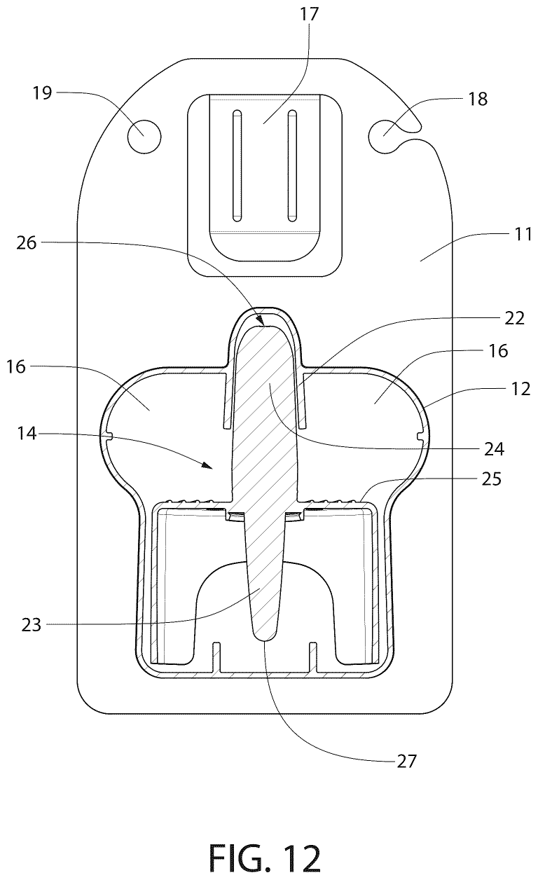

Referring now in detail to the various figures, wherein like reference numerals refer to like parts throughout, a blister pack 10 is shown. The blister pack 10 of FIGS. 4-12 is configured to hold the device 14 of FIGS. 1-3 for administering a drug. In the illustrated examples, the device 14 is a single dose nasal inhaler. Single dose nasal inhalers, such as the single dose nasal inhaler of FIGS. 1-3, are shown and described in U.S. Pat. No. 11,439,779, which is incorporated by reference herein in its entirety. The device 14 is a safe and effective mechanism for administering nasal inhalant treatments to patients. Its construction and intuitive operation is particularly well-suited for single dose administration of medication to a patient in emergency situations, such as when the patient is unconscious due to an acute medical condition.

In the illustrated embodiments, the drug delivery device 14 is a single dose nasal inhaler. Optionally, the device 14 is an inhaler, such as but not limited to nasal drug delivery devices, nasal inhalers, metered-dose inhalers (MDIs), dry powder inhalers (DPIs), and soft mist inhalers (SMIs). The device can thus be any from a wide range of medication administration devices and medications. However, a preferred device for storage in the blister pack 10 according to the disclosed concept is the drug delivery device 14 in the form of a nasal inhaler device as described herein and as illustrated in the accompanying drawing figures.

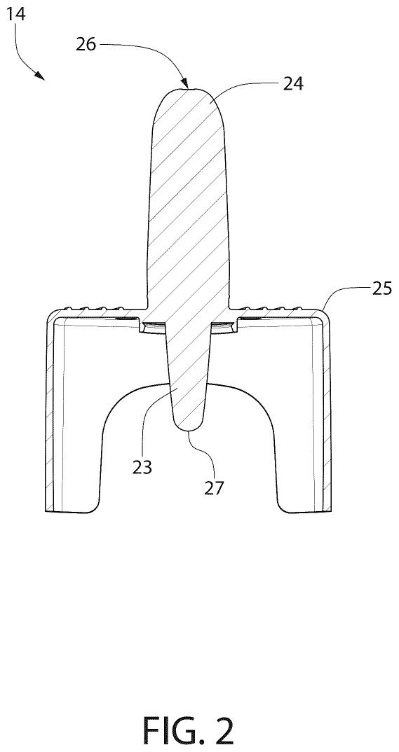

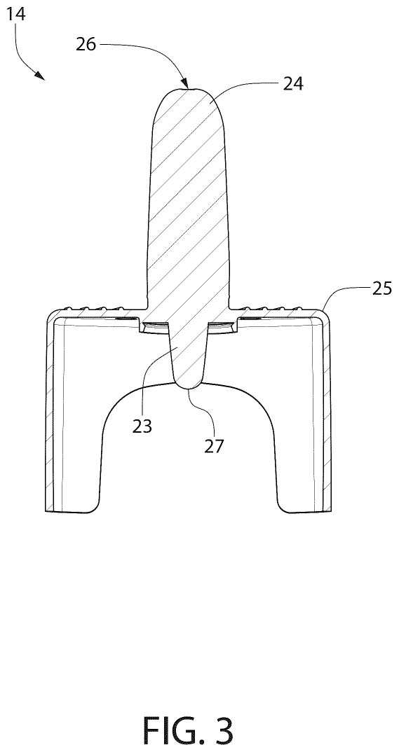

In the illustrated embodiment shown in FIGS. 1-3, 8 and 12, the drug delivery device 14 comprises an actuator body 23 connected to a discharge head 24 defining a drug discharge port 26. The actuator body 23 includes an actuator end 27 opposite the drug discharge port 26. The drug delivery device 14 further includes a drug reservoir having a drug stored therein. The drug delivery device 14 further includes a flange 25, each wing of the flange 25 extending radially from a respective side of the discharge head 24. The actuator body 23 is linearly movable relative to the discharge head 24 and flange 25, from an extended position (see FIG. 1) to a fully depressed or actuated position (see FIG. 3). FIG. 2 shows a partially depressed or actuated position as the actuator body 23 is being linearly advanced towards the discharge head 24. The discharge port 26 is sealed when the actuator body 23 is in the extended position and the discharge port 26 is open when the actuator body 23 is in the depressed position. The actuator body 23 is thus operable to discharge the drug from the reservoir upon axial movement of the actuator body 23 from the extended position to the depressed position. Optionally, to operate the drug delivery device 14, a user would insert the discharge head 24 into a nostril of a patient. The user would also place a finger on each wing of the flange 25 and a thumb on the actuator end 27. The user would then press the fingers towards the thumb (and/or vice versa) to axially move the actuator body 23 from the extended position to the depressed position, thereby dispensing the drug through the discharge port 26 into the patient's nostril.

The device 14 can optionally include a spring to facilitate movement (e.g., expansion and contraction) of the device 14 from an activated position in which at least some of the contents of the device 14 are released or administered, to a non-activated position in which contents of the device 14 are prevented from exiting. However, the device that may be stored in the blister pack 10 is not limited to being used as an inhaler; optionally, the device can be any structure configured to store, release, and/or administer contents held therein, such as an eyedropper or a pepper sprayer.

Optionally, the active pharmaceutical ingredient (API) stored within and/or delivered by the device 14 can be one or more of Naloxone, Ketamine, Midazolam, Fentanyl, Lorazepam, Glucagon, Nalmefene, Epinephrine, Apomorphine, Cetrorelix, Loxapine, Eletriptan, Ketorolac, and/or Olanzapine or any pharmaceutically acceptable salts of a member(s) selected from the foregoing.

The device 14 may be pre-filled and provide a medication in dosage form. In particular, the device 14 can be a dispersion device that delivers a medication in a dispersed form, such as in metered aerosol or spray devices. The device 14 can be pre-filled with the medication(s) in a finished dosage form. Embodiments include devices such as referenced in 68 Fed. Reg. 36,675, 36,676 and 36,680 (Jun. 18, 2003). Exemplary embodiments include intranasal aerosol and spray devices.

The blister pack 10 comprises a cover 11. The cover 11 defines a cavity 12 for receiving the drug delivery device 14. The cavity 12 is formed by injection molding. The cavity 12 is dimensioned to receive a drug delivery device 14 therein. The cavity 12 can be cylindrical, spherical, or of any suitable shape for providing a receptacle into which the drug delivery device 14 can stably be inserted and stored.

In the illustrated embodiments, the cover 11 and the cavity 12 are a single, unitary piece. The cavity 12, as shown, is in substantially the same profile as the drug delivery device 14. However, covers or cavities according to the disclosed concept may be other shapes, e.g., rectangular cuboid, and thus have more than a single continuous (e.g., round) sidewall.

A backing 13 is disposed on a rear surface of the cover 11. The backing 13 forms a seal with the cover 11 to form a sterile housing for the drug delivery device 14. In the illustrated embodiments, the seal follows the contour of the cavity 12, such as to minimize the amount of space in which the drug delivery device 14 is stored, and further prevent damage from movement of the drug delivery device 14 within the cavity 12.

In the illustrated embodiments, the backing 13 is removable from the cover 11 to allow access to the cavity 12 and the drug delivery device 14 stored therein. In the demonstrated embodiments, at least one tab 20 is provided to assist a user in removing, e.g., peeling, the backing 13 from the cover 11. In the specific embodiments shown, four tabs 20 are defined on the backing 13, consisting of a pair of upper tabs and a pair of lower tabs. The pair of upper tabs, as shown, are relatively elongated and extend upward from the upper edge of the backing 13. The pair of lower tabs are defined at the bottom edge of the backing 13 at the bottom corners of the cover 12. Furthermore, in the illustrated embodiments, each tab 20 comprises an indicator to assist the user in visual identification of each tab 20. Optionally, the number of tabs may vary, e.g., the backing 13 may include only one or two tabs, if desired.

Optionally, in the illustrated embodiments, the cavity 12 further comprises a sleeve 22. The sleeve 22 is configured to further secure and restrict movement of the drug delivery device 14. Specifically, in the embodiments of the blister pack 10 configured to secure a single dose nasal inhaler, the sleeve 22 is dimensioned to receive the discharge head 24 of the single dose nasal inhaler. The sleeve 22 at least partially surrounds a portion of the discharge head 24 and optionally provides an interference fit with the portion of the discharge head 24 when the sleeve 22 receives the discharge head 24.

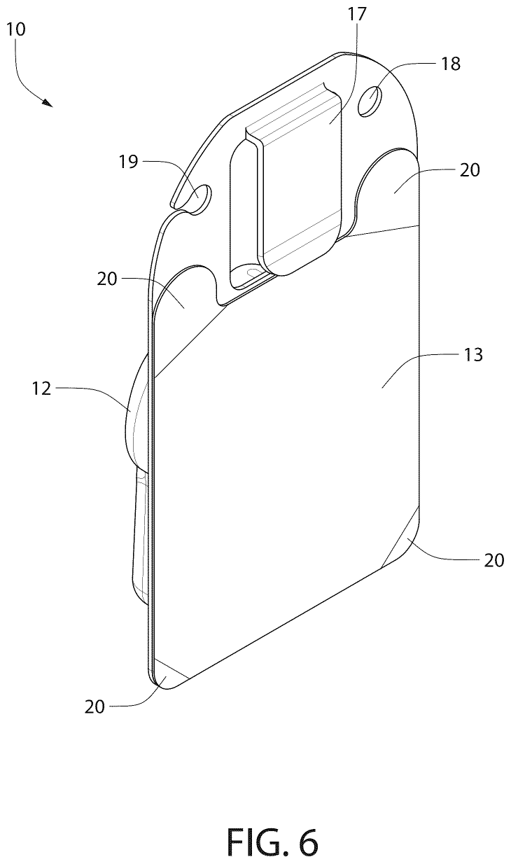

Furthermore, in the optional embodiment illustrated in FIGS. 6-9, the cover 11 comprises a plurality of ribs 21 extending into the cavity. The plurality of ribs 21 are configured to provide additional strength to the cover 11 and the protection to the drug delivery device 14. The ribs 21 are optionally linear and oriented horizontally (generally parallel) across a portion of the cover 11 on the outer surface of a product housing (shaped by the cavity 12).

Preferably, the cover 11 is rigid in construction, meaning that it retains its shape under gravity and through normal manual handling. Optionally, the cover 11 is made of polypropylene. Optionally, the cover 11 is made of polyethylene. The cover 11 may be of any suitable construction for providing crush-resistance to the cavity 12. Optionally, the cover 11 comprises a thickness from 1 mm to 2 mm. In a further embodiment, the cover 11 comprises a uniform thickness of 1 mm. In yet another embodiment, the cover 11 comprises a uniform thickness of 2 mm. As such, the cavity will be rigid, crush-resistant, and puncture-resistant, maintaining sterility of the drug delivery device 14. Optionally, the cavity 12 has a maximum depth of 2 cm to 5 cm, a maximum width of 5 cm to 10 cm and a maximum height of 15 to 22 cm.

In the illustrated embodiment, the cavity 12 defines a pair of finger receptacles 16. The pair of finger receptacles 16 is dimensioned to receive the fingers of the individual removing the drug delivery device 14 from the cavity 12. Using the pair of finger receptacles 16 to remove the drug delivery device 14 from the cover 11 reduces the risk of inadvertent actuation of the drug delivery device 14.

Furthermore, in the illustrated embodiment, the cover 11 optionally comprises a belt clip 17. The belt clip 17 extends outward from the rear face of the cover 11 and is substantially parallel to the cover 11 but occupying a different plane from that of the cover. As such, the belt clip 17 can secure the blister pack 10 to a belt or other suitable location for the user that is readily accessible. This feature may be beneficial to emergency medical personnel who may quickly need to administer lifesaving medication to a patient in need. Additionally, in the illustrated embodiment, the cover 11 comprises a pull-away slot 18 formed in the cover 11. The pull-away slot 18 is configured to enable quick removal of the blister pack 10 from a storage location. Furthermore, in the illustrated embodiment, the cover 11 comprises a permanent carrying aperture 19. The permanent carrying aperture 19 is configured to secure the blister pack 10 to a desired structure.

In use as a method of treating a patient using a drug delivery device 14 stored in the blister pack 10, the backing 13 is removed from the cover 11. Optionally, the backing 13 is removed from the cover 11 by peeling the backing 13 off the cover 11 from one or more of the tabs 20 defined on the backing 13. The drug delivery device 14 is removed from the cavity 12 of the cover 11. For example, the drug delivery device 14 may be removed by a user placing his or her fingers within the pair of finger receptacles 16 and grasping the flange of the actuator 25 of the drug delivery device 14. The discharge head 24 of the drug delivery device 14 is inserted into a nostril of a patient. The drug delivery device 14 is actuated to intranasally administer the drug to the patient.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the present presently disclosed technology.

What is claimed is:

1. A blister pack configured to hold a device for administering a drug, the blister pack comprising:
a cover defining a cavity for at least partially receiving the device for administering a drug, the cavity having been formed by injection molding; and
a backing disposed on a rear surface of the cover, forming a seal therewith.

2. The blister pack of claim 1, wherein the cover is rigid.

3. The blister pack of claim 1, wherein the cover is composed of polypropylene or polyethylene.

4. The blister pack of claim 1, wherein the cover has a thickness of from 1 millimeter to 2 millimeters.

5. The blister pack of claim 1, wherein the cavity of the cover defines a pair of finger receptacles, wherein each finger receptacle is disposed on an opposing side of where the device would be positioned when the device is disposed within the cavity.

6. The blister pack of claim 1, further comprising a member formed in the cover, the member selected from the group consisting of: a belt clip, a pull-away slot and a permanent carrying aperture.

7. The blister pack of claim 1, wherein the seal follows the contour of the cavity.

8. The blister pack of claim 1, wherein the backing comprises at least one tab configured to assist a user in peeling the backing from the cover, enabling access to the cavity to extract the device.

9. The blister pack of claim 1, wherein the device for administering the drug is disposed in the cavity and is a single dose nasal inhaler.

10. The blister pack of claim 9, wherein the single dose nasal inhaler comprises an actuator body connected to a discharge head defining a drug discharge port, the actuator body including an actuator end opposite the drug discharge port, the single dose nasal inhaler including a drug reservoir having a drug stored therein, the single dose nasal inhaler further including a flange extending radially from each side of the discharge head, the actuator body being linearly movable relative to the discharge head and flange, from an extended position to a depressed position, the discharge port being sealed when the actuator body is in the extended position and the discharge port being open when the actuator body is in the depressed position, the actuator body being operable to discharge the drug from the reservoir upon axial movement of the actuator body from the extended position to the depressed position.

11. The blister pack of claim 10, wherein the cavity of the cover comprises a pair of finger receptacles, wherein each finger receptacle is defined adjacent to the flange, to enable a user to place one finger into each finger receptacle to remove the single dose nasal inhaler from the cavity.

12. The blister pack of claim 1, wherein the cavity defines a plurality of inwardly extending ribs that structurally reinforce the cover in the area of the cavity to resist crushing.

13. The blister pack of claim 11, wherein the cavity defines a sleeve that receives a portion of the discharge port of the single dose nasal inhaler to secure and restrict movement of the single dose nasal inhaler in the blister pack during storage.

14. A medication delivery system comprising:
a. a blister pack configured to hold a nasal inhaler device for administering a drug, the blister pack comprising:
i. a cover defining an injection molded cavity for at least partially receiving the nasal inhaler device for administering a drug, the cover being rigid, being composed of polypropylene or polyethylene and having a thickness of from 1 millimeter to 2 millimeters; and
ii. a backing disposed on a rear surface of the cover, forming a seal therewith; and
b. a nasal inhaler device for administering a drug stored within the blister pack, the nasal inhaler device comprising an actuator body connected to a discharge head defining a drug discharge port, the actuator body including an actuator end opposite the drug discharge port, the nasal inhaler device including a drug reservoir having a drug stored therein, the nasal inhaler device further including a flange extending radially from each side of the discharge head, the actuator body being linearly movable relative to the discharge head and flange, from an extended position to a depressed position, the discharge port being sealed when the actuator body is in the extended position and the discharge port being open when the actuator body is in the depressed position, the actuator body being operable to discharge the drug from the reservoir upon axial movement of the actuator body from the extended position to the depressed position;
wherein the cavity of the cover comprises a pair of finger receptacles, wherein each finger receptacle is defined adjacent to the flange, to enable a user to place one finger into each or either finger receptacle to remove the single dose nasal inhaler from the cavity.

15. The medication delivery system of claim 14, wherein the cavity defines a sleeve that receives a portion of the discharge port of the nasal inhaler device to secure and restrict movement of the single dose nasal inhaler in the blister pack during storage.

16. The medication delivery system of claim 14, wherein the backing comprises at least one tab configured to assist a user in peeling the backing from the cover, enabling access to the cavity to extract the nasal inhaler device.

17. The medication delivery system of claim 14, wherein the nasal inhaler device is configured for intranasal administration and the drug, when administered, is in the form of a liquid, an aerosol, a gel or a powder.

18. The medication delivery system of claim 17, wherein the drug is a formulation comprising one or more of the following active pharmaceutical ingredients or pharmaceutically acceptable salts thereof: Naloxone, Ketamine, Midazolam, Fentanyl, Lorazepam, Nalmefene, Epinephrine, Apomorphine, Cetrorelix, Loxapine, Eletriptan, Ketorolac, Glucagon and Olanzapine.

19. A method of treating a patient in need of a drug that is administered intranasally, the method comprising:
providing the medication delivery system of claim 14;
peeling away the backing from the cover;
removing the nasal inhaler device from the cavity formed in the cover;
inserting the discharge head into the nostril of the patient; and
actuating the nasal inhaler device to intranasally administer the drug to the patient.

20. The method of claim 19, wherein removing the nasal inhaler device from the cavity includes placing a finger into each finger receptacle and squeezing the fingers together to grasp the nasal inhaler device and lift the nasal inhaler device from the cavity.

21. The method of claim 19, wherein removing the nasal inhaler device from the cavity includes placing a finger into one of the finger receptacles to reach under the nasal inhaler and then lift the nasal inhaler device from the cavity.

22. The method of claim 19, wherein the nasal inhaler device is configured for intranasal administration and the drug, when administered, is in the form of a liquid, an aerosol, a gel or a powder.

23. The method of claim 22, wherein the drug is a formulation comprising one or more of the following active pharmaceutical ingredients or pharmaceutically acceptable salts thereof: Naloxone, Ketamine, Midazolam, Fentanyl, Lorazepam, Nalmefene, Epinephrine, Apomorphine, Cetrorelix, Loxapine, Eletriptan, Ketorolac, Glucagon and Olanzapine.

24. The method of claim 23, the formulation comprising Naloxone or a pharmaceutically acceptable salt thereof, the patient suffering from opioid overdose, the method comprising administering a therapeutically effective amount of the Naloxone or pharmaceutically acceptable salt thereof to treat the patient's opioid overdose.

25. The method of claim 24, wherein the formulation comprises Naloxone hydrochloride.

26. The method of claim 23, the formulation comprising Epinephrine or a pharmaceutically acceptable salt thereof, the patient suffering from allergic reaction or asthma, the method comprising administering a therapeutically effective amount of the Epinephrine or pharmaceutically acceptable salt thereof to treat the patient's allergic reaction or asthma.

27. The method of claim 23, the formulation comprising Glucagon or a pharmaceutically acceptable salt thereof, the patient suffering from hypoglycemia, the method comprising administering a therapeutically effective amount of the Glucagon or pharmaceutically acceptable salt thereof to treat the patient's hypoglycemia.
